(19) United States
(12) Patent Application Publication
(10) Pub. No.: US 2006/0134725 A1
(43) Pub. Date: Jun. 22, 2006

(54) Method for the determination of the presence of an antibiotic in a fluid

(76) Inventor: Pieter Cornelis Langeveld, Delft (NL)

Correspondence Address: NIXON & VANDERHYE, PC, 901 NORTH GLEBE ROAD, 11TH FLOOR, ARLINGTON, VA, 22203, US

(21) Appl. No.: 10/559,785

(22) PCT Filed: Jul. 1, 2004
(86) PCT No.: PCT/EP04/07293

§ 371 Date: Dec. 8, 2005

(30) Foreign Application Priority Data
Jul. 2, 2003 (EP) 03077073.9

(51) Int. Cl. C12Q 1/18

(52) U.S. Cl. 435/32

(57) ABSTRACT

The present invention provides a method for determining the presence or absence of an antibiotic in a fluid comprising: (a) contacting a fluid sample with a test medium comprising a test microorganism and at least one indicator; (b) incubating the test microorganism with the fluid under conditions whereby growth of the test microorganism occurs if no antibiotic is present in the fluid sample; and (c) detecting any growth or inhibition of growth of the test microorganism as appropriate by means of an indicator, characterized in that the ratio of the volume of said fluid sample to the volume of test medium exceeds 2:3 (0.68:1) (v/v). The invention further provides a kit suitable for determining the presence or absence of an antibiotic in a fluid comprising: (a) at least one container partially filled with a test medium comprising a test microorganism, at least one gelling agent and at least one indicator, and; (b) a device for adding fluid to the test medium, said device having a volume that exceeds a ratio of 2/3 (0.68:1) of the volume of the test medium.

### FIELD OF THE INVENTION

The present invention relates to an improved novel microbiological test method for the determination of the presence of antibacterial compounds in fluids such as milk, meat juice, serum and urine.

### BACKGROUND OF THE INVENTION

Microbiological test methods for the determination of antibacterial compounds, particularly residues of antibiotics such as cephalosporin, penicillin, tetracycline and derivatives thereof and chemotherapeutics such as sulfa's, in fluids such as milk, meat juice, serum and urine are known. Examples of such tests have been described in CA 2056581, DE 3613794, EP 0005891, EP 0285792, EP 0611001, GB A 1467439 and U.S. Pat. No. 4,946,777. These descriptions all deal with ready to use tests that make use of a test organism and will give a result by the change indicated by an indicator molecule; for instance a change of color of a pH- and/or redox-indicator, added to the test system. A change in the indicator indicates the presence of a growing test organism. The principle is that when an antibacterial compound is present in a fluid in a concentration sufficient to inhibit growth of the test organism the color of the indicator will stay the same, while, when no inhibition occurs, growth of the test organism is accompanied by the formation of acid or reduced metabolites or other phenomena that will induce an indicator signal.

The known test systems mentioned above include a test medium, such as an agar medium, inoculated with a suitable test organism, preferably a strain of Bacillus or Streptococcus, and a pH indicator and/or a redox indicator. The suitable test organism and the indicator are introduced into an optionally buffered agar solution, optionally nutrients are added to the solution and optionally substances which change the sensitivity to certain antimicrobial compounds in a positive or a negative way are added to the solution. Finally the agar solution is allowed to solidify to form the test medium in such a way that the test organisms stay alive but cannot multiply because of lack of nutrients and/or low temperature. Of course a suitable test should have the desired sensitivity with regard to the compounds to be tested for.

The problem with the test systems currently distributed on the market and/or described in the literature is that they do not provide a simple procedure by which the sensitivity towards certain analytes can be adapted. For example, a given test system may give an indicator change when the concentration of the analyte tested, e.g. penicillin G, exceeds a certain detection threshold value, e.g. 4 ppb. However, when certain (local) requirements prescribe a different threshold, or existing thresholds are changed for other reasons, said test systems cannot be easily adapted to a new threshold value. There is thus a need for an improved test method that may not have this problem.

Whereas all of the microbiological test methods for the determination of antibacterial compounds known in the art advocate certain amounts of fluid sample to be used in the test and have certain amounts of solidified test medium present in the test, the ratio of fluid sample to test medium is lower than 2:3 (0.6:1) (v/v), in general lower than 1:2 (0.5:1) (v/v). For instance, from the examples of EP 0611001, dealing with the detection of antibiotics in milk, it becomes clear that a standard test medium volume is 0.3 ml onto which 0.1 ml of fluid sample may be brought. Likewise, in U.S. Pat. No. 4,946,777, 0.2 ml of fluid sample is contacted with 0.5 ml of test medium. The commercially available Delvotest® has two different designs, one using 0.1 ml of fluid sample on 0.27 ml of test medium, the other using 0.1 ml of fluid sample on 0.15 ml of test medium. The prior art documents and commercially available test methods in practice never disclose a ratio of fluid sample to test medium exceeding 2:3 (v/v). Also, none of them indicate that the value of said ratio, or the amount of fluid sample per se, has an effect on the sensitivity of the test system.

### SUMMARY OF THE INVENTION

It is an object of the present invention to provide an improved method for determining the presence or absence of antibiotics in fluids. Surprisingly, we have found that there is a remarkable effect attainable when applying increased volumes of fluid to be tested to the test medium.

The invention provides a method for determining of the presence or absence of an antibiotic in a fluid comprising:
(a) contacting a fluid sample with a test medium comprising a test microorganism, and at least one indicator;
(b) incubating the test microorganism with the fluid under conditions whereby growth of the test microorganism occurs if no antibiotic is present in the fluid sample; and
(c) detecting any growth or inhibition of growth of the test microorganism as appropriate by means of an indicator,
   characterized in that the ratio of the volume of the fluid sample to the volume of the test medium exceeds 2:3, such as 0.68:1 (v/v).

The invention further provides a kit suitable for determining the presence or absence of an antibiotic in a fluid comprising:
(a) at least one container partially filled with a test medium comprising a test micro-organism, at least one gelling agent and at least one indicator, and;
(b) a device for adding fluid to the test medium, said device having a volume that exceeds a ration of 2/3 (0.68:1) of the volume of the test medium.

Finally, there is provided the use of a ratio of volume of fluid sample to test medium between 2:3 (0.68:1) (v/v) and 10:1 (v/v) to improve the sensitivity of a test microorganism to β-lactams.

### DETAILED DESCRIPTION OF THE INVENTION

The terms and abbreviations given below are used throughout this disclosure and are defined as follows.

The term ‘CFU’ is an abbreviation of Colony Forming Units and refers to the number of microorganisms, spores of microorganisms, partially germinated spores of microorganisms or vegetative cells capable of producing colonies of microorganisms.

The term ‘fluid’ refers to a substance (as a liquid, not a gas) tending to flow or conform to the outline of its container.

The term ‘gelling agent’ refers to a compound that assists in changing a mixture into or taking on the form of a gel.

The term ‘indicator’ refers to a substance used to show (for example by change of color or fluorescence) the condition of a mixture such as a solution or a gel with respect to the presence of a particular material (for example an acid, a base, oxidizing or reducing agents). For instance, the term ‘indicator’ may refer to one or more compounds that are known as pH-indicators, but also to one or more compounds that are known as redox-indicators. Also, the term ‘indicator’ may refer to mixtures of two or more different types of indicators, such as a combination of a pH- and a redox-indicator.

The term ‘nutrient’ refers to one or more nutritive substances or ingredients that promote and/or are required for the growth of microorganisms as used in the method of the present invention.

The term ‘ratio’ refers to the value obtained when the volume of fluid sample is divided by the volume of test medium and can be expressed either as a fraction (e.g. 2/3), but preferably in decimal form (e.g. at least 0.68).

The term ‘sampling device’ refers to a device with the aid of which a sample of a fluid can be added to a test medium. Such a device may be a container, optionally with volume markings. Such a container may be a capillary, a syringe, a pipette or an automated pipetting system. Such a syringe or pipette may be designed in such a fashion that with only one mode of operation a predetermined volume can be withdrawn from the fluid to be analyzed.

The term ‘spore’ refers to a primitive usually unicellular often environmentally resistant dormant or reproductive body produced by plants or microorganisms and which is capable of development into a new individual.

The term ‘test medium’ refers to a solid composition, preferably in the form of a sol or a gel, which may comprise a gelling agent. Suitable examples of gelling agents are agar, alginic acid and salts thereof, carrageenan, gelatin, hydroxypropylguar and derivatives thereof, locust bean gum (Carob gum), processed eucheuma seaweed and the like. However, the person skilled in the art will understand that other types of solid test media may be based on carrier materials such as ceramics, cotton, glass, metal particles, paper, polymers (in any shape or form), silicates, sponges, wool and the like. Usually, a test medium contains one or more indicators, however, these compounds may also be added during the test method. The test medium comprises one or more types of test microorganisms as detecting agents. Optionally, the test medium may also contain nutrients, stabilizers, and substances that change the sensitivity to certain antimicrobial compounds in a positive or negative way, and/or viscosity-increasing agents. Examples of substances that change the sensitivity to certain antimicrobial compounds are substances that improve the sensitivity of the test organism towards sulfa compounds, such as antifolates like ormethoprim, tetroxoprim and trimethoprim and substances that improve the sensitivity towards tetracycline, such as salts of oxalic acid or hydrofluoric acid. Examples of viscosity-increasing agents are ascorbyl methyl-silanol pectinate, carbomer, carboxymethyl cellulose, cetearyl alcohol, cetyl alcohol, cetyl esters, cocamide DEA, emulsifying wax, glucose, hydroxyethyl cellulose, hydroxy-propylmethyl cellulose, lauramide DEA, linoleamide DEA, magnesium aluminum silicate, maltodextrins, PEG-8 distearate, polyacrylamide, polyvinyl alcohol, PVP/hexadecene copolymer, sodium chloride, sodium sulfate, soyamidopropyl betaine, xanthan gum and the like. Alternatively, the optional ingredients of the test medium mentioned above may be added during the test method.

The term ‘threshold’ refers to the concentration value above which a given analyte is to be regarded as present and below which said analyte is to be regarded as absent. Generally, a threshold value is given for particular analytes in particular samples by local, regional or interregional authorities but it can also be pre-set for certain research purposes.

In a first aspect of the invention, there is provided a method for determining the presence or absence of an antibiotic in a fluid comprising the steps of contacting a fluid sample with a test medium comprising CFU's of a microorganism and at least one indicator. The system may also comprise nutrients. Preferably, the test medium is a sol or gel comprising a gelling agent and/or a carrier material. Advantageously, the method also provides for conditions whereby there is minimal growth of a microorganism prior to the addition of fluid sample. Such conditions comprise an unfavorable temperature and/or an unfavorable pH-value and/or the absence of nutrients essential for growth, provided these conditions do not cause irreversible damage to all CFU's present. After addition of the fluid sample, growth of the microorganism is allowed to take place during a period sufficiently long for the microorganisms to grow in case no antibiotic is present. Growth is encouraged by adding nutrients, optionally before the contacting of said fluid sample, and/or raising the temperature, and/or providing for a pH-value at which the microorganism is able to grow. Alternatively, these conditions may be established prior to contact of the fluid sample with the test medium. Growth of the microorganism is detected by observing the presence or absence of a change of the indicator, whereby the ratio of the fluid sample to test medium exceeds 2:3 (0.68:1) (v/v). Preferably, said ratio is at least 20:27 (0.74:1) (v/v), more preferably said ratio is at least 25:27 (0.93:1) (v/v); most preferably said ratio is at least 2:1 (v/v).

It has been found however that there appears to be no technical reasons for an upper limit to the amount of fluid sample. In practice this volume should not exceed the maximum content of the container that holds the test medium. For example, in a 2 ml container having 0.2 ml test medium, no more than 1.8 ml of fluid sample should be added. In practice, containers for performing the method of the present invention have a volume that rarely exceeds 50 ml and hence the amount of fluid sample to be added shall not exceed 50 ml, preferably 10 ml, more preferably 5 ml, still more preferably 2 ml, most preferably 1 ml. Thus, in general, the upper limit of the ratio of the volume of fluid sample to the volume of test medium is 250:1 (v/v), preferably 50:1 (v/v), more preferably 25:1 (v/v), still more preferably 10:1 (v/v), most preferably 5:1 (v/v). Preferably, the volume of fluid sample is greater than the volume of test medium. The method of the present invention also includes mixing samples,(e.g. with other samples, but also with salts, buffering compounds, nutrients, stabilizers, isotope-labeled compounds, fluorescence-labeled compounds and the like), concentrating and/or diluting (e.g. with diluting liquids such as water, milk or liquids derived from milk, blood or liquids derived from blood, urine and/or solvents) samples prior to addition to the test medium.

The amount of gelling agent in the test medium is between 2 and 100 g.l⁻¹, preferably between 5 and 50 g.l⁻¹, more preferably between 10 and 20 g.l⁻¹, most preferably between 12 and 15 g.l⁻¹.

In one embodiment of the present invention, the antibiotic is a β-lactam antibiotic such as a cephalosporin or a penicillin derivative. Examples of such derivatives are amoxicillin, ampicillin, cefadroxil, cefradine, ceftiofur, cephalexin, penicillin G, penicillin V and ticarcillin, but of course many other similar β-lactam derivatives are known and applicable in the method of the present invention. Advantageously, it was established that the method of the present invention displays selectivity with regard to β-lactam antibiotics. The sensitivity for these compounds could be improved whilst simultaneously the sensitivity for chemotherapeutics such as sulfa's remained virtually unchanged. This phenomenon is of utmost importance in test systems where a change of sensitivity for one analyte is called for, whereas the sensitivity for another analyte is already satisfactory, a situation that occurs quite frequently in practice.

The effect of using the specific ratio of the volume of fluid sample to the volume of test medium according to the method of the present invention is surprising. For instance, when testing for an antibiotic such as penicillin G in fluids such as milk, it has been established that by doubling the amount of fluid sample advocated by the producer of the test, surprisingly the sensitivity towards penicillin G increases by a third. This implies that the method of the present invention either results in lower sensitivity thresholds, or that existing thresholds are reached faster or more accurately. Finally, it appears that upon increasing the amount of fluid sample to be applied on the test system, a considerable improvement of the test system could be achieved. In this respect, particular reference is made to test methods that make use of incubation at elevated temperatures and wherein such incubation is performed in heating devices that do not compensate for loss of moisture due to evaporation. This may result in unwanted irregularities in the test medium that are overcome by the method of the present invention.

The ratios of the present invention exceed those that are known from the prior art. A priori, the person skilled in the art would not use volumes larger than those advocated in the prior art. The reasons for this are manifold. Firstly, an increase of the sample volume could mean an increase in unwanted contaminants that could negatively influence the test. Secondly, as the sample volume is increased, the buffering capacity of the test medium might be lowered. Thirdly, increasing the amount of sample increases the pressure on the test medium and as a consequence thereof part of the sample could invade between the wall of the container and the test medium; this is phenomenon leads to difficulties in observation of the test and is particularly problematic with colored and non-transparent samples.

In another embodiment of the method of the invention, the test microorganism is incubated for a predetermined period, preferably within a time span of 0.5 to 4 hours, more preferably between 0.75 to 3 hours, most preferably between 1.0 to 2.75 hours.

Preferably the microorganism is incubated at a predetermined temperature, preferably the optimal growth temperature of the microorganism. When, for example, thermo stable microorganisms are used, said temperature is preferably between 40 and 70° C., more preferably between 50 and 65° C., most preferably between 60 and 64° C. Optionally said reaction can be carried out with the aid of a thermostatic device. Alternatively, the time required for growth of the microorganism is equal to the time that is required for a calibration sample with a known amount of analyte(s) to induce a change in the indicator.

In yet another embodiment of the method of the invention, the microorganism is a thermo stable microorganism such as a Bacillus species, preferably Bacillus stearothermophilus, or Streptococcus species, preferably Streptococcus thermophilus. These species may be introduced in the test as units capable of producing colonies, or Colony Forming Units (CFU's). Said CFU's may be spores, vegetative cells or a mixture of both. The concentration of said CFU's is expressed as Colony Forming Units per ml of test medium (CFU.ml⁻¹) and is usually in the range of 1×10⁵ to 1×10¹² CFU.ml⁻¹, preferably 1×10⁶ to 1×10¹⁰ CFU.ml⁻¹, more preferably 2×10⁶ to 1×10⁹ CFU.ml⁻¹, most preferably 5×10⁶ to 1×10⁸ CFU.ml⁻¹, or still more preferably 5×10⁶ to 2×10⁷.

In still another embodiment of the method of the invention, nutrients are added as a separate source, e.g. as a tablet, disc or a paper filter. Also other compounds such as the indicator(s), stabilizers and/or antifolates may be added as a separate source, or optionally incorporated in the nutrient medium.

At least one indicator is present during growth of the microorganism in the presence of the fluid sample in order to indicate any changes that take place in the reaction medium. The skilled artisan will appreciate that many indicators are suitable for this purpose. Particularly useful are indicators that, upon changing from one state to the other, provide a visually detectable signal such as a change in color or fluorescence. Such indicators may be easily selected from handbooks such as ‘H. J. Conn's Biological Stains’, R. D. Lillie ed., Baltimore, 1969. Preferred indicators are pH-indicators and/or redox indicators. Examples of suitable indicators are Acid Blue 120, Acid Orange 51, Acid Yellow 38, Alizarin acid, Alizarin Blue, Azure A, Azure B, Basic Blue 3, Brilliant Black, Brilliant Cresyl Blue, Brilliant Crocein MOO, Brilliant Yellow, Bromocresol Purple, Bromophenol Blue, Bromophenol Red, Bromothymol Blue, Congo Red, Gallocyanine, Indigo Carmine, Janus Green B, Litmus, Methylene Blue, Nile Blue A, Nitrazol Yellow (also referred to as Nitrazine Yellow), o-Nitrophenol, p-Nitrophenol, 1-10 Phenanthroline, Phenolphthalein, Safranine O, Thionin, Toluidine Blue.

The presence or absence of an antibiotic is determined by the presence or absence of a change of the indicator or indicators used. When, for example such a change is a color change, said color change may be observed visually. However in one embodiment of the invention said color change is determined using an arrangement that generates digital image data or an arrangement that generates analog image data and converts said analog image data into digital image data followed by interpretation of said digital image data by a computer processor. Such an arrangement, which may for instance be a sample-reading device such as a scanner coupled to a personal computer, is described in International Patent Application WO 03/033728, incorporated by reference, and briefly summarized below.

The arrangement can be used for detecting residues of antibiotics in milk. The commercially available Delvotest® and BR®-test are commonly used. Delvotest® comprises an agar matrix, CFU's of an acid forming microorganism, as well as a color indicator. With the arrangement mentioned above it is possible to automatically scan the bottom side of each of the samples in a test plate. The color and the brightness of the reflected light are registered in three variables, each describing one color component, for instance the so-called L*a*b* model. In the L*a*b* model, the color spectrum is divided in a two-dimensional matrix. The position of a color in this matrix is registered by means of the two variables “a” and “b”. The variable L indicates the intensity (for instance, from light blue to dark-blue). It is possible to make a criterion comprising the a-value, b-value and L-value to make a composite function as follows:
Z=w_{L}.L+wₐ.a+w_{b}.b

where w_{L}, wₐ and w_{b} are weighting factors for the L-value, a-value and b-value, respectively. The values of these weighting factors can be calculated by means of “discriminent analysis”, such that the group mean shows a maximum distance in relation to the spreading. By combining two or more of the color components in the L*a*b* model in a predetermined manner that depends on the type of residue and the sample, an accurate detection is possible. In practice, a certain value of Z at which a test should switch between positive and negative result (the threshold value) can be experimentally predetermined.

The invention further provides a kit for carrying out the method of the present invention. Such a kit comprises one or more containers filled with test medium as described in the method of the invention and a sampling device. The containers may be test tubes of any shape and size and of any material available, provided that observation of indicator changes is possible. Also, the containers may be wells such as those incorporated in micro-titer plates.

Said sampling device is a device with the aid of which fluid can be added to said test medium. Preferably, such a device is a container, optionally with volume markings. More preferably, such a device is a syringe, a pipette or an automated pipetting system. Such a syringe or pipette may be designed in such a fashion that with only one mode of operation a predetermined volume can be withdrawn from the fluid to be analyzed. Optionally, systems known in the art with which more than one syringe or pipette can be operated with one single handling may be applied. It is the object of the second aspect of the present invention to provide a kit that allows for simple addition of the amounts of fluid to be added according to the method of the invention. The method of the present invention is most easily and accurately carried out when the sampling device is designed such that the ratio of the volume of fluid sample to volume of test medium exceeds 2:3 (0.68:1) (v/v). Preferably, said ratio is at least 20:27 (0.74:1) (v/v); more preferably said ratio is at least 25:27 (0.93:1) (v/v); most preferably said ratio is at least 2:1 (v/v). For instance, in the commercially available test kit Delvotest® comprising containers with 0.27. ml of test medium, the present invention provides a sampling device that delivers, upon applying it only once, more than 0.19 ml of fluid, preferably 0.25 ml or more. In a preferred embodiment, said sampling devices are designed to give exactly said volumes. With exactly is meant an amount that is equal to the amount mentioned including an error range of less than 20%, preferably less than 10%, more preferably less than 5%. The person skilled in the art will understand that test kits comprising containers having other volumes of test medium which differ from the Delvotest® kit, similar sampling devices can be added that conform to the same ratio requirement as mentioned above.

Optionally, said kit comprises a means for sealing of said containers filled with test medium during incubation and/or an insert with instructions for use and/or a means for setting the time needed for incubation.

In one embodiment of the invention, said kit also comprises nutrients. Preferably said nutrients are contained within a medium such as a tablet, disc or a paper filter. The advantages of providing nutrients contained within a medium are that the user can easily add them to the test medium. The amounts can be predetermined so as to avoid errors in dosing the required amounts. Also other compounds such as the indicator(s), stabilizers and/or antifolates may be added as a separate source, or optionally incorporated in the nutrient medium.

In another embodiment of the present invention, said kit comprises a thermostatic device, with the aid of which test samples can be kept at a pre-set temperature. The temperature may be one at which the microorganism shows sufficient growth. Preferably, said thermostatic device is designed in such a fashion that it can hold said containers filled with test medium. Optionally the thermostatic device is coupled to a means for setting the time needed for incubation such that heating and/or cooling is stopped after lapse of a pre-set period.

In a further embodiment of the invention, said kit comprises a data carrier loaded with a computer program suitable for instructing a computer to analyze digital data obtained from a sample-reading device. Said data carrier may be any carrier suitable for storing digital information such as a CD-ROM, a diskette, a DVD, a memory stick, a magnetic tape or the like. Advantageously, said data carrier loaded with a computer program also provides for easy access to the latest available computer programs suitable for use in the method of the present invention.

The present invention further provides the use of a ratio of the volume of fluid sample to the volume of test medium exceeding 2:3 (0.68:1) (v/v), to improve the sensitivity of a test microorganism to β-lactams. Preferably said ratio is between 2:3 (0.68:1) (v/v) and 100:1 (v/v), more preferably between 2:3 (v/v) and 50:1 (v/v), most preferably between 2:3 (v/v) and 10:1 (v/v). Examples of fluid samples are fluids obtainable from the human or animal body such as milk, meat juice, serum and urine. Suitable β-lactams are cephalosporin and penicillin derivatives. Examples of such derivatives are amoxicillin, ampicillin, cefadroxil, cefradine, ceftiofur, cephalexin, penicillin G, penicillin V and ticarcillin.

### LEGEND TO THE FIGURES

FIG. 1 shows the relationship between the concentration of penicillin G (x-axis, in ppb) and the Z-value (y-axis) in a test with nutrients added in tablet form. As outlined in the detailed description, the following equation was used: Z=0.35.a+0.65.b. The explanation of the symbols used in the Figure is as follows.

◯: 0.30 ml of milk on 0.27 ml of test medium (ratio is 1.11:1);

Δ: 0.20 ml of milk on 0.27 ml of test medium (ratio is 0.74:1);

⋄: 0.10 ml of milk on 0.27 ml of test medium (ratio is 0.34:1);

□: 0.05 ml of milk on 0.27 ml of test medium (ratio is 0.19:1).

FIG. 2 shows the relationship between the concentration of penicillin G (x-axis, in ppb) and the Z-value (y-axis) in a test with nutrients added in tablet form. As outlined in the detailed description, the following equation was used: Z=0.35.a+0.65.b. The explanation of the symbols used in the Figure is as follows.

◯: 0.10 ml of milk on 0.27 ml of test medium, incubation time 2.4 h (ratio is 0.37:1);

Δ: 0.30 ml of milk on 0.27 ml of test medium, incubation time 2.4 h (ratio is 1.11:1);

⋄: 0.30 ml of milk on 0.27 ml of test medium, incubation time 3:0 h (ratio is 0.37:1);

□: 0.30 ml of milk on 0.27 ml of test medium, incubation time 3.0 h (ratio is 1.11:1).

FIG. 3 shows the relationship between the concentration of penicillin G (x-axis, in ppb) and the visual observation of the color (y-axis) wherein a numerical value is given ranging from a full color change (−3) to complete color conservation (+3). In this test, the nutrients were present in the test medium. The explanation of the symbols used in the Figure is as follows.

◯: 0.05 ml of milk on 0.15 ml of test medium (ratio is 0.33:1);

Δ: 0.10 ml of milk on 0.15 ml of test medium (ratio is 0.67:1);

⋄: 0.15 ml of milk on 0.15 ml of test medium (ratio is 1:1);

□: 0.20 ml of milk on 0.15 ml of test medium (ratio is 1.33:1);

♦: 0.30 ml of milk on 0.15 ml of test medium (ratio is 2:1).

### EXAMPLES

### Example 1

### Sensitivity of a Microbiological Test Method with Different Volumes of Milk in a Test System with Nutrients Added in the Form of a Tablet

A commercially available microbiological test system (Delvotest®) having 0.27 ml of test medium containing agar, CFU's of Bacillus stearothermophilus var. calidolactis, an antifolate, and the indicator Bromocresol Purple was investigated using milk with added penicillin G in concentrations of 0, 2, 3, 4 and 6 ppb. These five milk samples were applied to the test systems in a series of four different volumes, ie. 0.05 ml, 0.10 ml (the Delvotest® recommended volume), 0.20 ml and 0.30 ml. Before addition of the milk, a tablet with nutrients was applied on the test medium. After the milk was added, the tests were incubated for 2.6 hours at 64° C. If there are no or little antibiotics that inhibit the growth of the test organism, after some time, an acid environment is formed by the growing microorganisms and the indicator changes color from blue/purple to yellow. However, if there are sufficient antibiotics to inhibit growth, the color of the indicator remains purple.

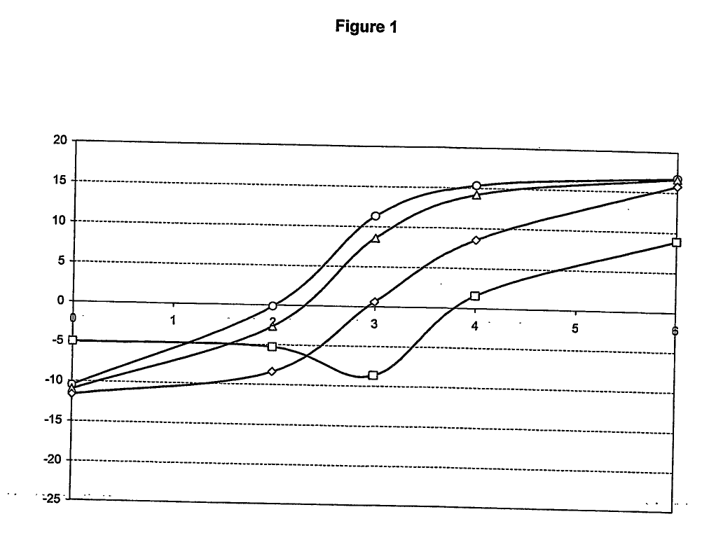

Firstly, the colors were measured using the scanning apparatus described in the detailed description using the following formula for the Z-value: Z=0.35.a+0.65.b. The results as graphically represented FIG. 1, show that sensitivity increases with increasing milk volume. If, for example, a Z-value of 0 is chosen as the required threshold value, it can be seen that, whereas the standard recommended amount of 0.10 ml milk gives a Z-value of 0 at a concentration of 3 ppb penicillin G, for 0.20 ml milk this threshold already is reached at 2.3 ppb and in case of 0.30 ml of milk at 2.0 ppb penicillin G. Secondly, upon visual inspection of the experiments, similar results were obtained. With 0.10 ml of milk a color change was observed between concentrations of 3 and 4 ppb penicillin G, with 0.20 ml milk between concentrations of 2 and 3 ppb penicillin G, and with 0.30 ml of milk between concentrations of 1 and 2 ppb penicillin G. The enhanced sensitivity of the method using volumes of 0.20 ml or 0.30 ml is sustained throughout the range of penicillin G concentrations tested.

### Example 2

### Sensitivity of a Microbiological Test Method with Different Volumes of Milk at Different Time Intervals in a Test System with Nutrients Added in the Form of a Tablet

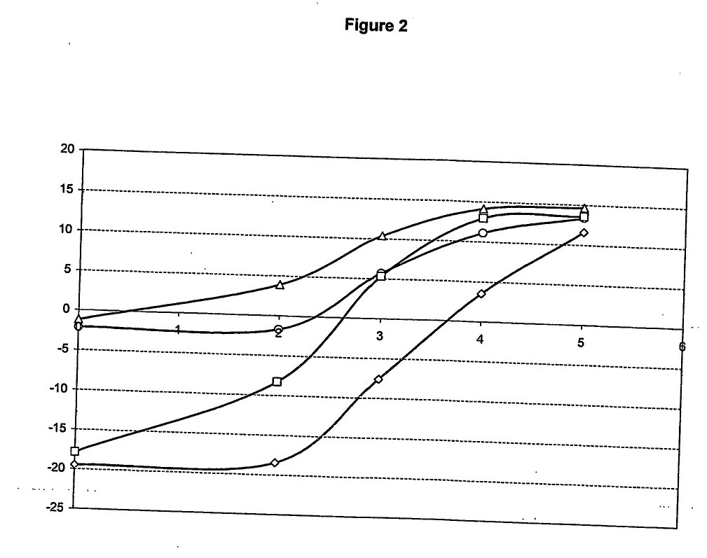

A similar experimental setup as in Example 1 was used. However, in this case the penicillin G concentrations were 0, 2, 3, 4 and 5 ppb and the volumes of milk that were investigated were 0.10 ml and 0.30 ml. Test systems were incubated at 64° C. (for 2.4 h, normally referred to as the point of indicator change, and 3.0 h, normally referred to as the point of reading). The results, as graphically represented in FIG. 2, again clearly show that sensitivity increases with increasing milk volume (compare Δ with ◯ and □ with ⋄). Furthermore, it becomes clear from FIG. 2 that an increase in milk volume leads to stable results over time and in the concentration range tested. With this is meant that the result at, for instance, 4 ppb penicillin G with 0.30 ml of milk after 2.4 h (see line Δ at 4 ppb: Z=14) is almost the same as after 3.0 h (see line □ at 4 ppb: Z=13). This has the advantage that tests now show an increased robustness with regard to small errors in timing of incubation made during the application. In the daily dairy practice, such small errors are easily made. In sharp contrast, the result at 4 ppb penicillin G with 0.10 ml of milk differs considerably between 2.4 h (see line ◯ at 4 ppb: Z=11) and 3.0 h (see line ⋄at 4 ppb: Z=4).

### Example 3

### Sensitivity of a Microbiological Test Method with Different Volumes of Milk in a Test System with Nutrients Present in the Test Medium

A commercially available microbiolgical test system (Delvotest®) having 0.15 ml of test medium containing agar, CFU's of Bacillus stearothermophilus var. calidolactis, an antifolate, nutrients, and the indicator Bromocresol Purple was investigated using milk with added penicillin G in concentrations of 0, 0.5, 1, 2, 3 and 4 ppb. These six different milk samples were applied to the test systems in a series of five different volumes, i.e. 0.05 ml, 0.10 ml (the Delvotest® recommended volume), 0.15 ml, 0.20 ml and 0.30 ml. After the milk was added, the tests were incubated for 3 hours at a temperature of 64° C. If there are no antibiotics (or only little) that inhibit the growth of the test organism, after a certain amount of time, an acid environment is formed by the growing microorganisms. Then, the color of the indicator changes from blue/purple to yellow. However, if there are sufficient antibiotics to inhibit that growth, the color of the indicator does not change and remains purple.

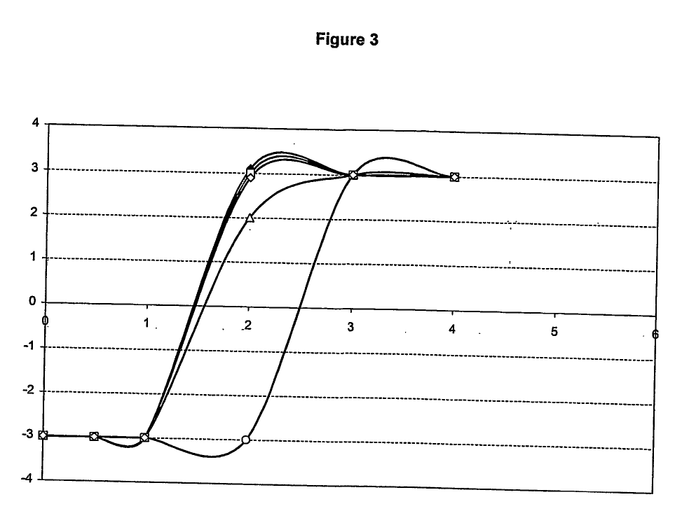

The colors were measured visually by assigning a value of −3 to a sample where full color change from blue/purple to yellow occurred, to a value of +3 where no color change was observed at all. Intermediate color changes were assigned values of −2, −1, 0, +1 or +2, depending on the degree of coloration. The results, as graphically represented in FIG. 3, clearly show that sensitivity increases with increasing milk volume. If, for example, a visual color of 2 is chosen as the required threshold value, it can be seen from FIG. 3 that, whereas the standard recommended amount of 0.10 ml milk gives a color of 2 at a concentration of 2 ppb penicillin G, whereas for 0.15, 0.20 and 0.30 ml milk this threshold already is reached at 1.7 ppb penicillin G.

1. A method for determining the presence or absence of an antibiotic in a fluid comprising:
(a) contacting a fluid sample with a test medium comprising a test microorganism and at least one indicator;
(b) incubating the test microorganism with the fluid under conditions whereby growth of the test microorganism occurs if no antibiotic is present in the fluid sample; and
(c) detecting any growth or inhibition of growth of the test microorganism as appropriate by means of an indicator,
characterized in that the ratio of the volume of said fluid sample to the volume of test medium exceeds 0.68:1.

2. A method according to claim 1, wherein the antibiotic to be determined is a β-lactam antibiotic.

3. A method according to claim 1, wherein the test medium is a sol or a gel.

4. A method according to claim 1, wherein the indicator is at least one pH-indicator and/or at least one redox-indicator.

5. A method according to claim 1, wherein the test microorganism is a thermo-stable microorganism.

6. A method according to claim 1, wherein in the fluid sample comprises a fluid obtainable from an animal or human body.

7. A method according to claim 6, wherein the fluid is milk.

8. A method according to claim 1, wherein the conditions for growth of the test micro-organism comprise:
(a) adding nutrients; and/or
(b) incubating at an appropriate temperature; and/or
(c) incubating for a sufficient period of time.

9. A method according to claim 1, wherein the ratio of the volume of liquid sample to the volume of test medium exceeds 20:27 (0.74:1) (v/v), 25:27 (0.93:1) (v/v) or 2:1 (v/v).

10. A method according to claim 1, wherein the volume of liquid sample is greater than the volume of test medium.

11. A kit suitable for determining the presence or absence of an antibiotic in a fluid comprising:
(a) at least one container partially filled with a test medium comprising a test micro-organism, at least one gelling agent and at least one indicator, and;
(b) a device for adding fluid to the test medium, said device having a volume that exceeds a ratio of 2/3 (0.68:1) of the volume of the test medium.

12. A kit according to claim 11 further comprising nutrients suitable for allowing the microorganism to grow.

13. A kit according to claim 11, further comprising a thermostatic device, with the aid of which test samples can be kept at a pre-set temperature.

14. A kit according to claim 11, further comprising a data carrier loaded with a computer program suitable for instructing a computer to analyze digital data obtained from a sample-reading device.

15. Use of a ratio of a volume of fluid sample to a volume of test medium between 2:3 (0.68:1) (v/v) and 10:1 (v/v) in an assay for the detection of an antibiotic.

16. Use of a ratio of a volume of fluid sample to a volume of test medium between 2:3 (0.68:1) (v/v) and 10:1 (v/v) to improve the sensitivity of a test microorganism to β-lactams.
